(19) United States
(12) Patent Application Publication
(10) Pub. No.: US 2008/0041399 A1
(43) Pub. Date: Feb. 21, 2008

(54) Sterile Enclosures

(76) Inventor: Hans Rudolf Kriek, Bussum (NL)

Correspondence Address: DIEDERIKS & WHITELAW, PLC, 12471 DILLINGHAM SQUARE, #301, WOODBRIDGE, VA, 22192, US

(21) Appl. No.: 10/594,180

(22) PCT Filed: Mar. 24, 2005
(86) PCT No.: PCT/GB05/01206

§ 371 Date: Jun. 15, 2007

(30) Foreign Application Priority Data
Mar. 24, 2004 (GB) 0406648.6

(51) Int. Cl. A61B 19/10; A61B 19/12

(52) U.S. Cl. 128/852; 128/849; 128/853; 128/856

(57) ABSTRACT

An inflatable sterile enclosure is disclosed comprising a flexible membrane and at least a pair of rigid or semi-rigid panels which are arranged to pivot outwardly relative to one another on each side of the enclosure as it is inflated. At least one of the panels is transparent and provides a window. The enclosure is preferably inflatable by a pressurised gas source. The flexible membrane may comprise a plurality of support members arranged to define a support frame for the enclosure when erected. The support members may themselves be inflatable. Peripheral sterile enclosures and surgical docks or drapes comprising bactericidal substances are also disclosed.

The present invention relates to apparatus and enclosures for use in sterile environments and, in particular, to an enclosure which may be used during operations and to which peripheral enclosures or attachments may be connected.

The maintenance of sterile environments during surgery on the human or animal body is of primary importance in preventing infections arising as a result of surgery and improving the success rate of surgical operations. Generally, surgical theatres are kept as clean as possible to prevent infections during surgery and, both for use in surgical theatres and outside of such environments, various apparatus and systems have been suggested for maintaining a sterile environment in which surgical procedures can be carried out. In particular, it has been proposed to perform operations in sterile enclosures, such as tents or clean rooms, the inside of which have previously been sterilised and can be kept clean. Such sterile enclosures have been proposed not only to allow performance of surgical operations outside of surgical theatres but also for use in surgical theatres and the like to reduce the time spent in sterilising the theatres and also generally to reduce the likelihood of infections arising.

It is an object of the invention to provide an improved sterile enclosure having an inflatable, flexible membrane.

According to one aspect of the present invention there is therefore provided an inflatable sterile enclosure comprising a flexible membrane and at least a pair of rigid or semi-rigid panels which are arranged to pivot outwardly relative to one another on each side of the enclosure as it is inflated, wherein at least one of the panels is transparent and provides a window.

This arrangement has the advantage that the panels may be closed together with the collapsed membrane fully or partly folded therebetween during transportation or storage. In use, the panels are folded outwardly as the membrane is inflated, and therefore add stability to the inflated structure as well as providing improved visibility to the interior of the enclosure through at least one rigid or semi-rigid transparent surface.

In the preferred embodiment, the sterile enclosure is an operating enclosure in which surgery may be performed.

In the preferred embodiment all of the panels are prefabricated window panels. These window panels may be positioned on the upper and/or side portions of the enclosure. The window panels preferably do not extend continuously along the entire length of the enclosure but are provided in co-operating pairs which pivot with respect to each other. The co-operating pairs of window panels may be hinged together or spaced slightly apart on a flexible portion of the enclosure. The individual window panels may be any shape and may be flat or curved as desired. In a preferred embodiment all the window panels can be folded to a position lying generally one above the other with the flexible membrane located therebetween and thereby protected in a compact configuration for storage or transportation.

The preferred operating enclosure comprises a flexible membrane in the form of a collapsible bubble, which may be of a plastics material and is preferably transparent. A collapsible bubble is particularly straightforward to manufacture such that its interior surface can conveniently be kept sterile and, as it may be supplied in a collapsed state with the bubble folded up between the rigid panels, it may be easily transported and stored.

In preparation for use, the enclosure may be filled with air or gas to inflate it such that it is suitable for the performance of a surgical procedure. Preferably, the enclosure is provided with a gas entry port through which gas or air may be passed to inflate the enclosure. In use, gas or air may be continually circulated within the enclosure to maintain it sterile or otherwise suitable for the operational procedure. Therefore, preferably a pump or other propulsion system and a sterilising filter are coupled to the enclosure to sterilise the air or gas. The air or gas which is used to inflate the enclosure or which is subsequently circulated within the enclosure may be provided with a bactericidal substance. In a particularly preferred embodiment a canister or source of pressurised air or gas is provided in communication with the inside of the enclosure and the pressurised air or gas may be released into the enclosure to inflate it. Preferably, parameters of the air or gas inside the enclosure which may affect the operational procedure are controlled. For example, the temperature and humidity of the air or gas may be regulated to control fluid evaporation inside the enclosure.

When the enclosure is inflated it may be sufficiently rigid to support itself. It may be required to inflate the enclosure such that the air or gas within it is at a pressure slightly above atmospheric pressure to help support the enclosure. The enclosure may also be suspended from a separate frame to keep it in the desired position during performance of the surgical procedure. However, in a particularly preferred embodiment the enclosure itself comprises elongate support members mounted to the flexible membrane which provide a supporting frame for the enclosure once it is inflated. The elongate support members extend around the edges of the membrane and may optionally be interconnected with one another to strengthen the support frame after inflation of the enclosure. The membrane may be secured to the outside or inside surface of the membrane. In some embodiments the support frame formed by the elongate membrane may be attached in use to a surgical trolley or bed on which the patient lies.

Viewed from a second aspect the invention provides a collapsible, sterile enclosure comprising a flexible membrane which can be inflated to define the interior of the enclosure, there being a plurality of elongate support members secured to the membrane, such members being arranged to define a supporting frame for the enclosure when erected.

In another embodiment at least some of the support members comprise inflatable means attached to the enclosure. Alternatively, the support members may comprise inflatable portions of the enclosure membrane. These inflatable support members provide for an operating enclosure which may be folded into a particularly compact configuration as such folding is not limited by the size, number or arrangement of the support members when they are deflated. Therefore, the preferred enclosure is especially convenient to store and transport. Furthermore, the enclosure may be expanded into its operating configuration automatically by inflating the support members. Inflation of the support members may be facilitated by sources of pressurized gas or other means.

Accordingly, viewed from another aspect the present invention provides a collapsible, sterile enclosure comprising a flexible membrane which can be inflated to define an interior of the enclosure, the membrane being provided with one or more inflatable support members, such members being arranged to define a supporting frame for the enclosure when erected.

When the operating enclosure is in use surgical instruments, tools, fluids, medicines, blood, saline, organs or other sterile objects may need to be introduced into the operating enclosure. These objects may be contained within and maintained sterile by a peripheral enclosure which can be attached to the operating enclosure in order to introduce the objects into it.

Therefore, according to a further aspect of the present invention, there is provided a closed, generally tubular sterile enclosure adapted to form a peripheral enclosure to a large sterile enclosure, comprising an end wall member having means for joining it to a wall of a larger enclosure, wherein a surface on or adjacent to the end wall member is provided with a bactericidal substance which sterilises the join formed between the enclosure in use, the peripheral enclosure containing one or more surgical instruments.

Preferably, the bactericidal substance is covered by a removable protective layer.

The preferred tubular peripheral enclosure provides a particularly flexible system for introducing surgical instruments into the operating enclosure as it may be joined to the operating enclosure at any desired position. Thus, a surgical procedure can be carried out in the operating enclosure and surgical instruments contained in a sterile peripheral enclosure can be introduced into the operating enclosure in which the surgical procedure is being performed without contaminating that sterile environment. For example, the peripheral sterile enclosure containing surgical instruments might be joined to the operating enclosure and in doing so the surfaces of the peripheral and operating enclosures at the joined portions are sterilised by the bactericidal substance. The adjacent portions of the wall members of the peripheral and operating enclosures may then be opened, for example by cutting, to connect the operating and peripheral enclosures and to allow access to the surgical instruments.

The peripheral enclosure of the preferred embodiment provides a reduced possibility of contamination of the sterile environments (e.g. operating and peripheral enclosures) when they are interconnected. This is firstly because the sterile environments are only connected by opening an interconnected region between the environments and neither environment is therefore open to the external environment at any time, and secondly because the outer surfaces of the wall members of the sterile environments at and/or within the join are provided with a bactericidal substance to sterilise the adjacent wall members before they are opened, such that any portion of the adjacent wall members which have been exposed to the external environment are sterilised before opening.

In a particularly preferred embodiment, the joining means comprises an adhesive. The adhesive may be provided on the end wall member of the peripheral enclosure as an adhesive tape or layer. Preferably, the adhesive has a backing layer which may be peeled off when it is required to join the sterile environments.

In an alternative embodiment the joining means comprises an adhesive provided on a flange portion which extends radially outward from the main body of the peripheral enclosure.

The end wall member of the peripheral enclosure within the joining means can comprise a sponge material impregnated with a bactericidal substance. The joining means or the region within the joining means may comprise a perforable membrane. For example, a section of the end wall member of the tubular peripheral enclosure may comprise a sponge or perforable membrane with an adhesive layer around its periphery. In this manner, as the operating and peripheral enclosures are joined together, the adjacent wall members of each enclosure are sterilised. The wall members may then be perforated to connect the two environments. A scalpel or the like may be used to facilitate the perforation which may include cutting through the sponge or film of the peripheral enclosure.

However, it is possible that using an easily perforable membrane may weaken the wall member of the peripheral enclosure. Thus, in a preferred embodiment, the means for joining the enclosures comprises a membrane having a cleft sealed by a weaker membrane. The weaker membrane may then be broken to connect the enclosures after the join has been made. This has the advantage of ensuring the opening is made in a desired place and the opening being easy to make without compromising the strength of the wall member of the enclosures.

The substance provided on the surfaces of the peripheral enclosure that meet with the other enclosure can comprise any bactericidal, bacteriological or sterilising agent and can be applied in a variety of ways. For example, a bactericidal gas may be contained in a capsule which is perforated as the side walls are joined such that the side walls that meet are sterilised. However, in a particularly preferred example, the substance provided on the surfaces that meet comprises a bactericidal substance, such as iodine, impregnated into the wall members, sponge, membrane and/or adhesive layer. Thus, for example, as the adhesive sticks to make the join, the surfaces which meet to make the join are sterilised.

Viewed from a still further aspect the invention provides a peripheral enclosure having a sterile interior adapted to be secured to a sterile operating enclosure and to permit communication between the enclosures without contamination, a connecting surface of the peripheral enclosure being provided with a sponge material impregnated with a bactericidal substance and arranged to sterilise inter-engaged portions of the enclosures in use.

As discussed above, a preferred aspect of the invention is the provision of peripheral items which can be secured to an operating enclosure using a bactericidal adhesive or adhesive tape. In the case of an enclosure including an inflatable, flexible membrane there can be difficulties providing enough back pressure to enable these items to be securely fastened to the membrane.

Viewed from a further aspect the invention therefore provides an operating enclosure having a flexible membrane wall, there being a closed attachment compartment of flexible material secured to the wall, the interior of the compartment being sterile, the compartment being pleated to enable pressure to be manually applied on a reverse face of the compartment and transmitted to its front face, whereby an item can be engaged under pressure with the front face and adhered thereto, without the need to access the sterile interior to apply such pressure.

In order to perform any surgical procedure it is also, of course, necessary to have access to the patient. Conventionally, a patient is covered with surgical drapes during an operation and a hole is left in the drapes around the area of the intended incision through which the surgical procedure is to be carried out. This area is cleaned and has a bactericidal substance applied to it to reduce the risk of infection after the incision has been made. However, infection can still occur due to contamination after the bactericidal substance has been applied or from the surrounding environment directly into the incision after it has been made.

Accordingly, from another aspect the present invention provides a surgical dock or drape having an upper surface and a lower surface, at least a portion of both the upper and lower surfaces comprising a bactericidal adhesive or adhesive tape for joining the operation dock or drape both to a patient and to an overlying operating enclosure in use.

Preferably, the operation dock/drape is attached to the operating enclosure by the bactericidal adhesive or adhesive tape on its upper surface after the dock/drape is attached to the patient by the bactericidal adhesive or adhesive tape on its lower surface and when the patient is ready for surgery. In this manner the bactericidal adhesive or adhesive tape adheres the operation dock/drape to the patient and to the operating enclosure in the region of the patient where the incision is intended to be made thereby sterilising those surfaces of the operating enclosure and the patient which will be cut through when an incision is made.

Viewed from another aspect, the invention provides a surgical dock or drape provided with means having a bactericidal substance arranged to sterilise in use, regions of an underlying patient and of an operating enclosure overlying the dock or drape.

The operation dock/drape may comprise a layer in which the central portion is a sponge impregnated with a bactericidal substance and arranged to engage both the patient and the operating enclosure. The outer portion of the operation dock/drape surrounding the sponge may comprise an adhesive or bactericidal adhesive on either or both of the enclosure and patient contacting surfaces. In one embodiment the outer portion of the operation dock/drape comprises separate docking and patient layers for adhering to the operating enclosure and patient respectively. Preferably, the docking layer is relatively rigid and the patient layer is flexible so that it can conform to the patient.

In another embodiment the operation dock/drape comprises a disinfecting layer having a sponge impregnated with a bactericidal adhesive and another layer having an opening. In use the sponge of the disinfecting layer protrudes through the layer having the opening such that in use it may contact both the patient and the enclosure. Preferably, the upper and lower surfaces of the operation dock/drape are covered prior to use by protective covering layers.

In an alternative embodiment, the member provided with a bactericidal substance is a membrane layer which may seal an opening in the operation dock/drape. The at least one membrane layer and/or the layer having the opening may comprise a bactericidal adhesive or adhesive tape.

According to the preferred method, the area of the intended incision may be prepared and treated with a bactericidal substance, and the operation dock/drape then adhered to the patient with the means provided with a bactericidal substance over the area of the intended incision. The operating enclosure may then be joined to the operation dock/drape and the dock/drape connected to the enclosure such that a surgeon can gain access to the area of the intended incision from inside the enclosure. Thus, the area of the intended incision is not exposed to anything but a sterile environment after preparation for surgery.

From yet another aspect the present invention provides an operating dock/drape having an inflatable portion which surrounds the area of the incision in use.

In a preferred embodiment the inflatable portion may be provided on the surfaces which in use contact the patient and/or enclosure. Preferably, an inflatable portion or portions extend around the outer region of the operation dock/drape and may comprise an adhesive or bactericidal adhesive. In this manner, portions of the dock/drape may be inflated such that they provide a region to which the enclosure, and less preferably the patient, may be attached relatively easily. The dock/drape may also be provided with means which enable the inflatable portions to be deflated as desired.

In order to perform a surgical procedure, the surgeon must also, of course, have access to the sterile operating enclosure. In the prior art, gloves have been built into the wall of operating enclosures to receive a surgeons hands. However, these gloves often provide only restricted access to the enclosure as they are only provided at certain points on the operating enclosure and can therefore make surgery cumbersome and difficult by making it difficult to reach all areas of the enclosure easily. In order to overcome this problem glove carriers have been provided which may be attached to the desired region of the enclosure. However, it has proven difficult for the user to fit a pair of glove carriers over both hands. Therefore, the present invention provides an improved glove carrier.

Accordingly, from another aspect the present invention provides a glove carrier comprising a sterile glove region enclosed by a flexible membrane, wherein the flexible membrane comprises at least one exterior thumb or finger compartment alignable in use with a thumb or finger portion of the glove enclosed within the membrane.

Hence, the user may insert his or her hand into the glove, and subsequently insert a glove finger or, preferably, thumb into a corresponding compartment in the protective membrane of the glove carrier. The carrier itself therefore has a general glove or mitten configuration and can be used to manipulate other items, such as during placing a glove/glove carrier on the other hand. At all times the eventual outer surface of the glove within the glove compartment remains sterile.

Preferably, the exterior of the flexible membrane is provided with joining means proximal to the fingers of the glove portion. This enables the flexible membrane of the glove carrier to be attached to an operating enclosure. An opening is then formed in the joining means and through the wall of the operating enclosure and the surgeon can insert his hand within the glove portion into the enclosure, such that he can manipulate items within the operating enclosure. The glove carrier may therefore be connected to the operating enclosure where desired such that a surgeon can arrange easy access to the desired area(s) of the operating enclosure.

Various embodiments of the present invention will now be described, by way of example only, and with reference to the accompanying drawings, in which:

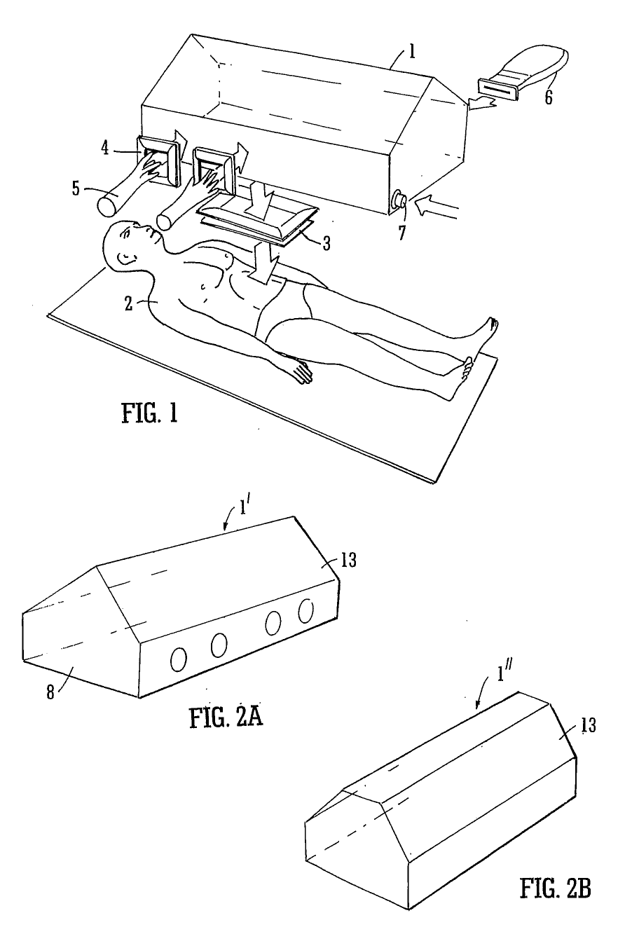

FIG. 1 is an illustration of an operating environment in accordance with a preferred embodiment of the invention;

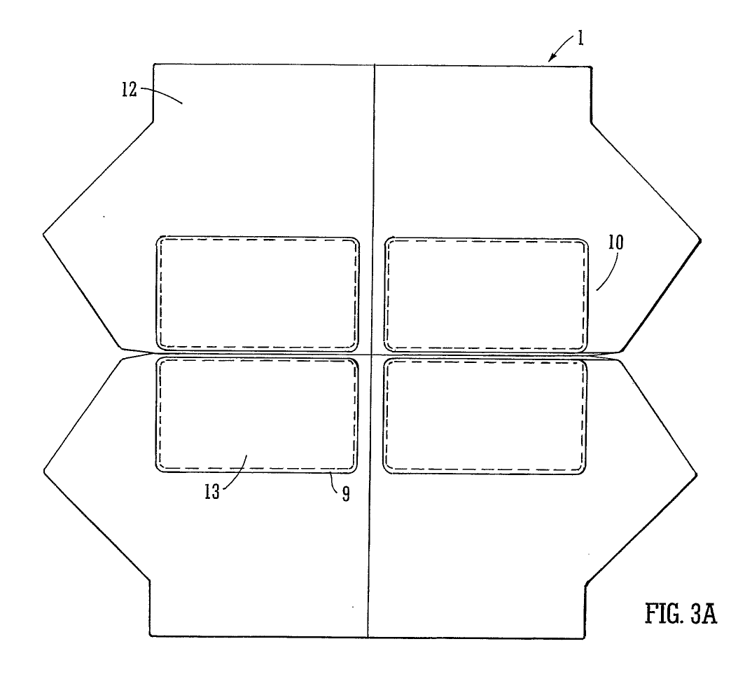

FIGS. 2A and 2B are illustrations of preferred embodiments of operating enclosures comprising different numbers of rigid panels;

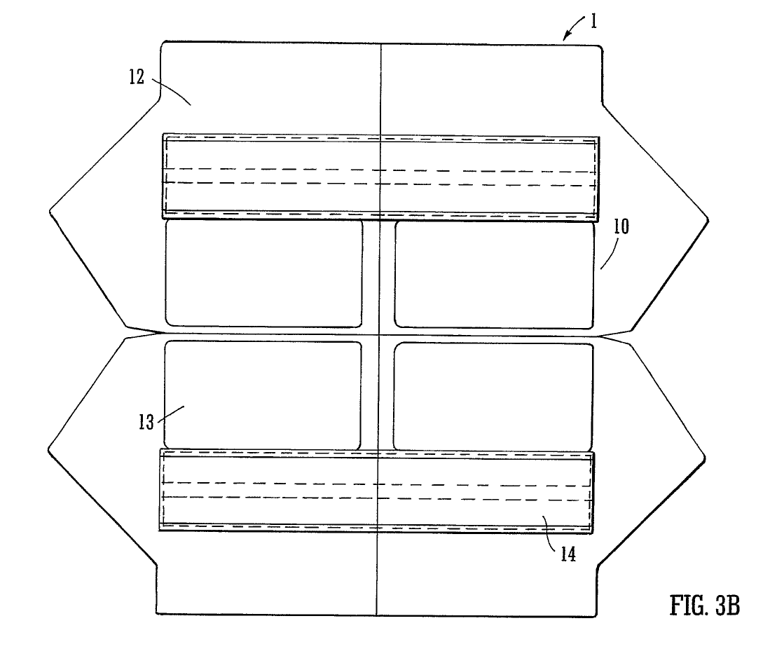

FIGS. 3A to 3F illustrate the preferred operating enclosure in various stages of its construction;

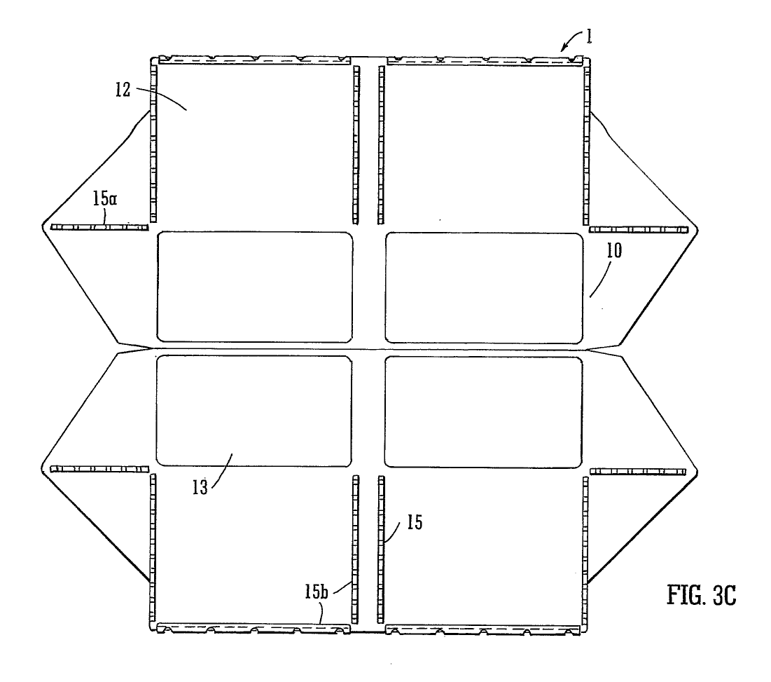

FIGS. 4A to 4C illustrate the preferred operating enclosure as it is unfolded ready for inflation;

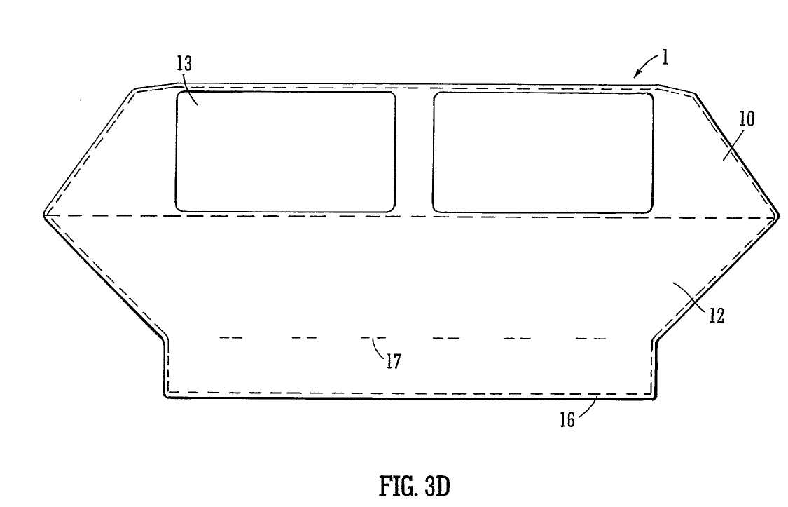

FIGS. 5A and 5B illustrate embodiments of preferred operating enclosures comprising flat windows and curved windows;

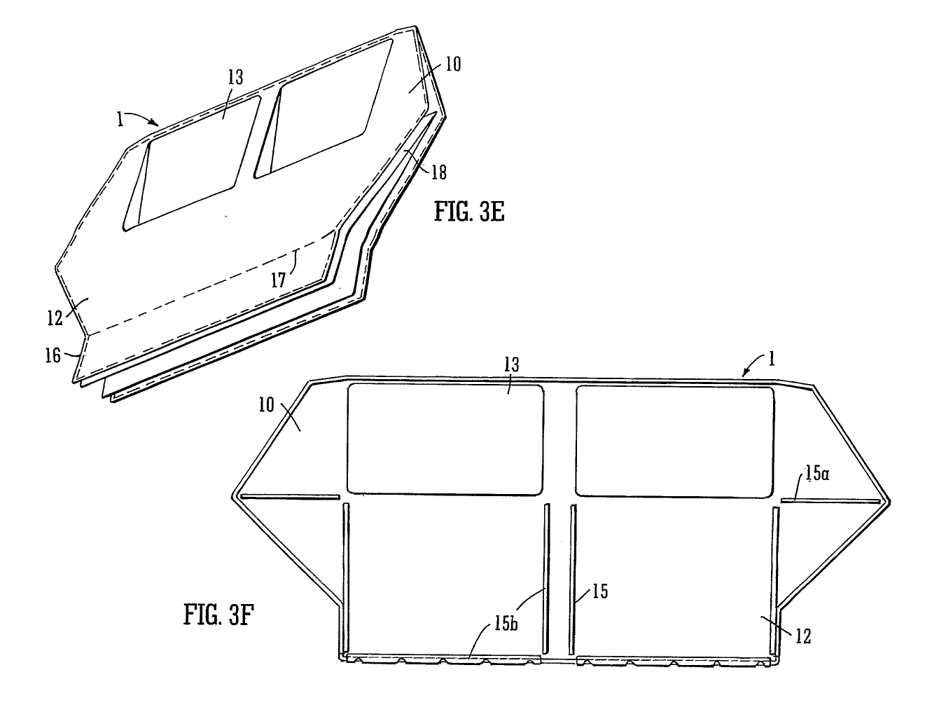

FIGS. 6A to 6C illustrate a portion of a preferred operating enclosure comprising support folds;

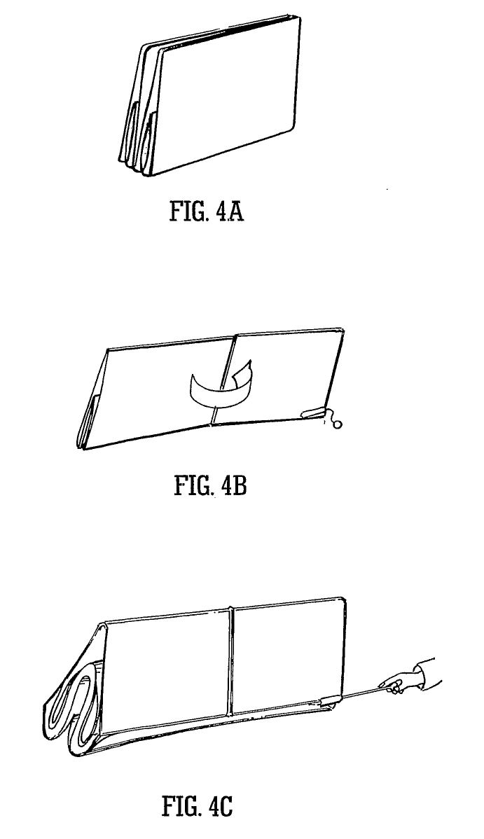

FIG. 7 illustrates a preferred operating enclosure attached to a patient and ready for use;

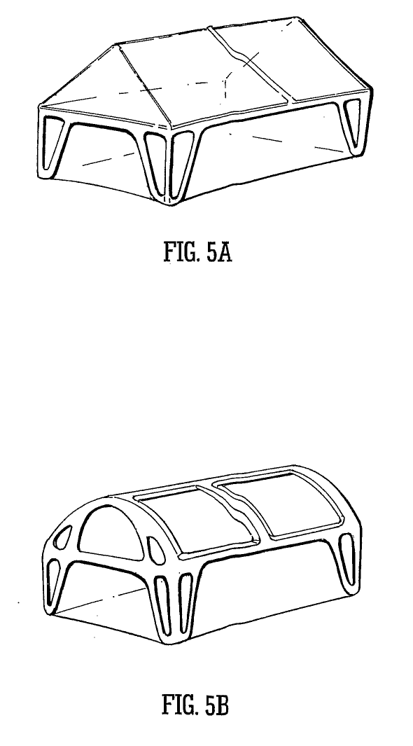

FIG. 8A illustrates a preferred operating enclosure comprising side compartments for attaching entrance/exit ports and FIGS. 8B and 8C illustrate methods of attaching entrance/exit ports to the side compartments;

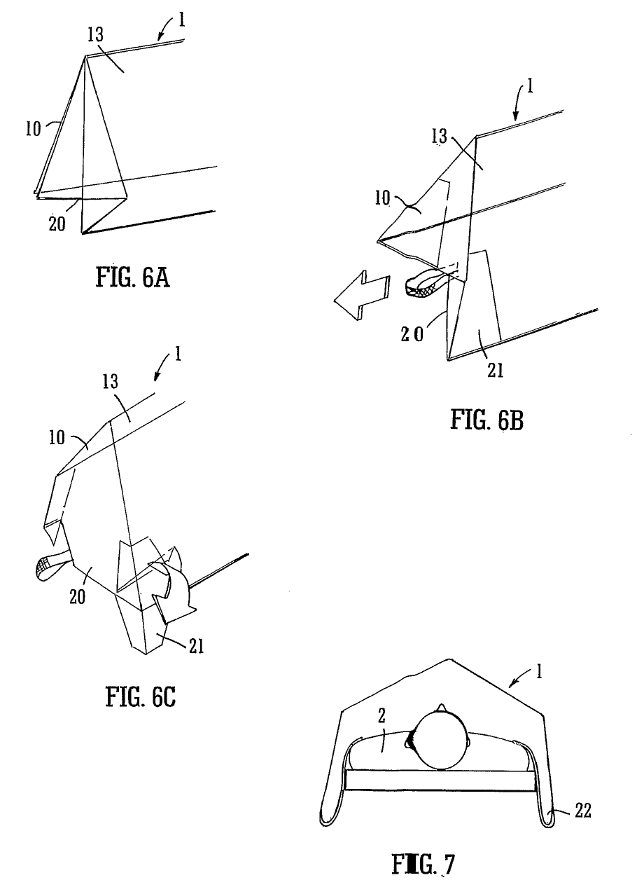

FIG. 9A illustrates a preferred embodiment of a glove carrier comprising a finger compartment on the exterior surface;

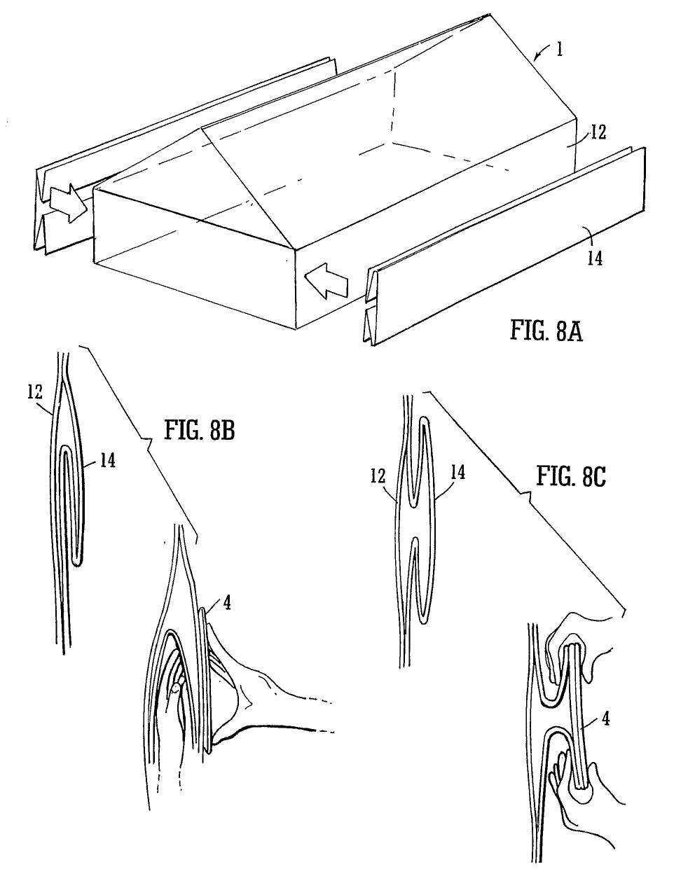

FIG. 10 illustrates a preferred embodiment of a peripheral enclosure;

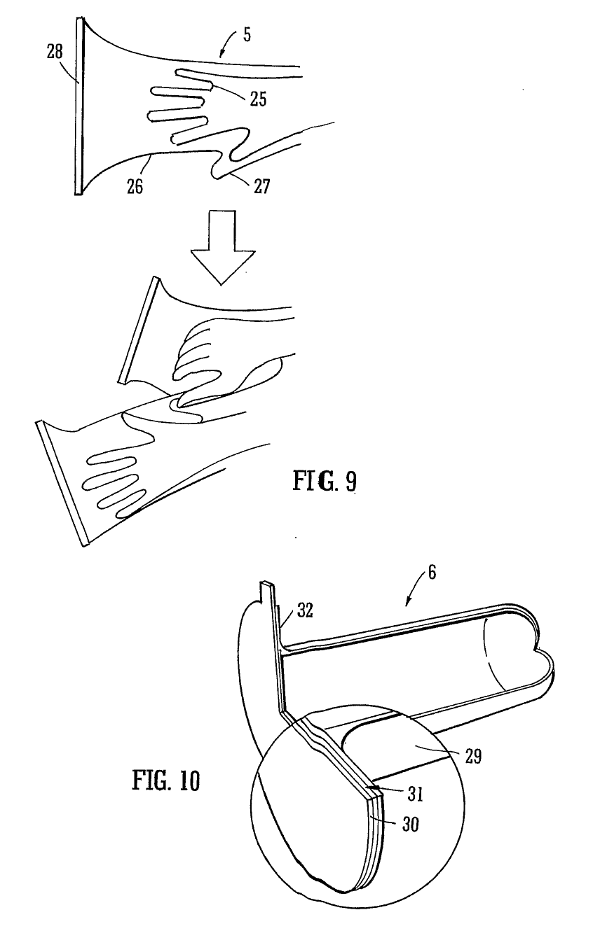

FIG. 11 illustrates a preferred embodiment of an operating dock or drape; and

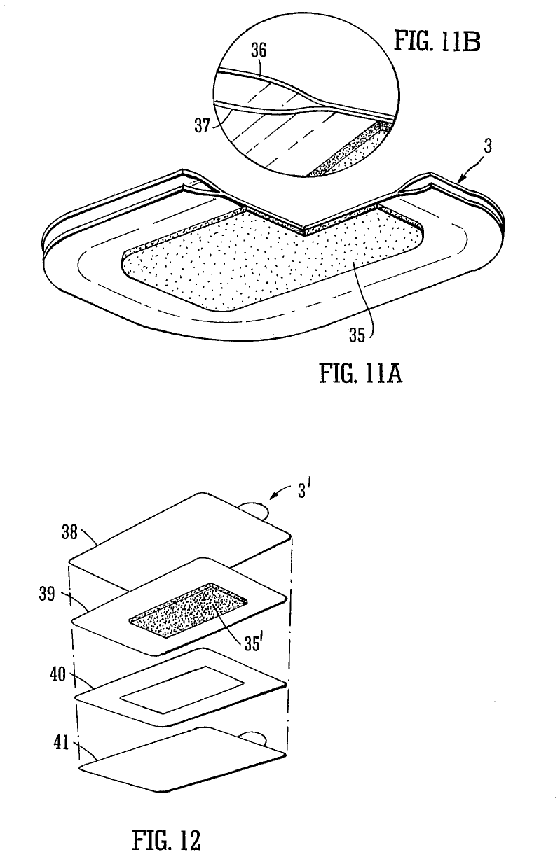

FIG. 12 illustrates a preferred embodiment of an operating dock or drape comprising a sponge impregnated with a bactericidal substance.

FIG. 1 shows an example of the apparatus which may be used to operate on a patient 2. The apparatus may comprise a sterile enclosure 1, an operating dock/drape 3, one or more entrance/exit ports 4 at which the operating team may access the enclosure 1 and one or more glove carriers 5 and peripheral enclosures 6.

In use the operation dock/drape 3 may be placed over or fixed to the region of the patient 2 at which the incision is intended to be made. The sterile operating enclosure 1 may then be attached to the operation dock/drape 3. Various attachments such as peripheral enclosures 6 and entrance/exit ports 4 may then be fixed to the operating enclosure 1. The operating enclosure 1 preferably consists of a flexible membrane and two or more rigid or semi-rigid panels and will be described in more detail below. The operating enclosure 1 is preferably inflatable and can be inflated before or after it has been attached to the operation dock/drape 3. The operating enclosure 1 may also comprise a gas entry port 7 which may be supplied with a gas to inflate the enclosure 1 and/or to circulate gas within the enclosure 1 during use. The gas supplied to the gas entry port 7 is preferably a sterilised gas such as air or a bactericidal gas. The composition of the gas may also be selected so as to control physiological parameters, for example, temperature or fluid balance.

FIGS. 2A and 2B show preferred embodiments of the operating enclosure 1′,1″. The operating enclosure 1,1″ comprises a flexible membrane comprising at least a pair of rigid or semi-rigid panels 13 which are pivotable in relation to each other and which may extend along the length of the operating enclosure 1′ as shown in FIG. 2A. In the embodiment shown in FIG. 2B the operating enclosure 1″ comprises three rigid or semi-rigid panels 13. At least one of the panels 13, more preferably all of the panels 13, are transparent and serve as windows to see into the operating enclosure 1′,1″. The operating enclosure 1′,1″ may be supported by maintaining the gas inside it at a pressure above atmospheric pressure. Alternatively, or in addition, the operating enclosure 1′,1″ may be partially or completely supported by the rigid panels 13 and/or by a support frame.

In a preferred embodiment, the window panels 13 need not extend continuously along the entire length of the operating enclosure 1. Preferably, two or more panelled sections 13 are provided on each side of the enclosure 1. The provision of multiple shorter window panels 13 on each side of the operating enclosure 1 enables it to flex longitudinally such that when the operating table or patient 2 is elevated or lowered the operating enclosure 1 is not undesirably distorted, broken or pulled away from the patient 2.

FIGS. 3A to 3F show schematics of the various steps in the construction of the preferred operating enclosure 1. FIG. 3A shows a plan view of the upper 10 and side 12 portions of the enclosure 1 in a completely unfolded state. Preferably, the upper 10 and side 12 portions of the operating enclosure 1 are formed of a flexible membrane. The window panels 13 may then be attached to the upper 10 and/or side 12 portions of the enclosure 1. The window panels 13 may be attached to the enclosure 1 by robotically controlled infra-red laser welding or by any other conventional means. Preferably, the windows 13 are attached to the enclosure 1 by an adhesive region 9 at the outer edge of the windows 13. The adhesive region 9 is preferably approximately 1 cm in width. Most preferably, the window panels are arranged in co-operating pairs along the length of the enclosure 1, each window 13 in a pair being pivotable with respect to each other. The windows 13 in each pair may be hinged together but are preferably pivotable as they are slightly spaced apart on the enclosure 1 with a region of flexible membrane between them, for example spaced apart by 2 cm. The pairs of windows 13 are also preferably spaced apart from each other along the length of the enclosure 1, for example by 8 cm. This region of flexible membrane between the pairs of windows 13 enables the enclosure 1 to flex longitudinally and hence accommodate elevation of the patient 2 without it being broken or pulled away from the patient. In the preferred embodiment, the membrane of the upper 10 and side 12 portions of the enclosure 1 is substantially transparent, although the portions of the membrane beneath the window panels 13 may be removed before or after the windows 13 are attached.

FIG. 3B shows a plan view of the upper 10 and side 12 portions of a preferred operating enclosure 1 in an embodiment where side compartments 14 have been attached to the outer surface of the side portions 12 of the enclosure 1. These side compartments 14 are provided to assist in the attachment of entrance/exit ports 4 to the enclosure 1 and will be described in more detail below. The side compartments 14 are preferably provided on the side portions 12 of the enclosure 1 below the window panels 13. The side compartments 14 may be attached to the enclosure 1 at approximately 34 cm from the longitudinal centre line.

FIG. 3C shows a plan view of the upper 10 and side 12 portions of a preferred operating enclosure 1 in an embodiment wherein structural support rods 15,15a,15b have been provided on the enclosure 1. In use, the support rods 15,15a,15b serve as a frame to support the enclosure 1 once it has been inflated. In the illustrated embodiment four support rods 15a are provided parallel to and just below the window panels 13. These support rods 15a may be positioned approximately 35 cm from the longitudinal centre line of the enclosure 1 and when the enclosure 1 is constructed for use the support rods 15a extend between the two side portions 12 of the enclosure 1. Further support rods 15b may be provided on each side portion 12 of the enclosure 1 to maintain the structure of the enclosure 1 when in use. Any number, configuration and length of support rods 15,15a,15b may be provided on the enclosure 1 to give the desired support frame.

In another embodiment, the support members comprise inflatable means. As described above in relation to the support rods 15,15a,15b any number, configuration and length of inflatable support members may be provided on the enclosure 1 to give the desired support frame. Advantageously the inflatable support members do not interfere with the folding of the enclosure 1 when deflated and therefore provide an enclosure 1 which can be folded more compactly and hence is more convenient to store and transport. In a particularly preferred embodiment a single, continuous inflatable support member may be provided which when inflated provides a support frame for the entire enclosure 1.

FIG. 3D shows a view of one of the upper 10 and side 12 portions of the enclosure 1 at a stage where the lower portion of the enclosure 1 is attached. A flexible membrane may be arranged in facing relationship to the inner surface of the upper 10 and side 12 portions of the enclosure 1. The flexible membrane is then preferably attached to the peripheral region of the upper 10 and side 12 portions of the enclosure 1 as shown by the dashed line 16. The flexible membrane may further be attached to the side portions 12 of the enclosure 1 as shown by the broken dashed line 17 in order that the lower surface of the enclosure 1 maintains a desirable configuration in use. Preferably, this attachment region 17 is intermittent and may be provided approximately 15 cm inward of the peripheral attachment 16.

FIG. 3E shows the completed enclosure 1 in a configuration where the lower portion 18 of the enclosure 1 which contacts the patient 2 in use has been folded up between the upper 10 and side 12 portions of the enclosure 1.

FIG. 3F shows a side view of the enclosure 1 in an embodiment having support rods 15,15a, 15b which act as a frame when the enclosure 1 is inflated. The number, arrangement and length of the support rods 15,15a,15b and window panels 13 can be selected so that the enclosure 1 can fold into any desired compact configuration.

FIGS. 4A to 4C show a preferred operating enclosure 1 comprising two pairs of window panels 13, the adjacent window panels 13 being hinged or pivotable with respect to each other. FIG. 4A shows the preferred operating enclosure 1 in its folded, compact configuration. The windows 13 in each pair are pivotable relative to each other about the apex of the operating enclosure 1 and about a transverse axis so that they can be folded into a condition lying one above the other. In this configuration the side 12 and lower 18 portions of the operating enclosure 1 have been folded up between the pairs of window panels 13. In embodiments where the side portions 12 of the enclosure 1 comprise support rods 15,15a,15b then these may be arranged and sized such that they may also be folded between the pairs of window panels 13. In this embodiment the pairs of window panels 13 are slightly spaced apart and/or hinged together so that the enclosure 1 may be folded longitudinally in order to reduce the length of the enclosure 1. As such, it can be seen that the preferred enclosure 1 provides for convenient carrying and storage.

FIG. 4B shows the preferred enclosure 1 when it has been unfolded to its full length. Once the enclosure 1 has been unfolded to its full length it may be expanded such that it is suitable for use. This may be achieved, for example, by inflating the enclosure 1. Alternatively, the process of inflating the enclosure 1 may unfold it to its full length. Preferably, the inflated length of the enclosure 1 is approximately 130 cm. However, the folded enclosure 1 may be much smaller, for example, 65 cm in length.

FIG. 4C shows a preferred embodiment in which the enclosure 1 is provided with a container of compressed gas 19, for example CO₂ or air, which may be released into the enclosure 1 to inflate it. Alternatively, gas may be supplied to the enclosure 1 at a pressure above atmospheric pressure through an inlet port 7. The gas may be sterilised or contain a bactericidal agent to sterilise the enclosure 1. Inflation of the enclosure 1 causes the lower 18 and side 12 portions of the enclosure 1 folded between the window panels 13 to expand. The rigid window panels 13 automatically pivot away from each other as the enclosure 1 inflates such that it expands to the desired shape. The enclosure 1 may be maintained in its expanded state by maintaining the pressure of the gas inside the enclosure 1 above atmospheric pressure and/or by a support frame. The enclosure 1 may be attached to a conventional support frame although preferably support members are provided on the enclosure 1. The support members may be interconnected to support the expanded enclosure 1.

Once the enclosure 1 is expanded sterile air or gas may be circulated within it. The air or gas may have a bactericidal substance added to it in order to maintain sterility of the enclosure 1. The gas or air may also be continually recirculated through a sterilising filter. A pump and filter arrangement may therefore be attached to the operating enclosure 1 for this purpose.

FIGS. 5A and 5B show embodiments of the enclosure 1′,1″′ after inflation and having flat and carved window panels 13 respectively. In these embodiments rigid support rods 15 (as shown in FIG. 3C) are provided on the side portions 12 of the enclosure 1 and may be interconnected after inflation to provide a support frame for the enclosure 1.

FIGS. 6A to 6C show a portion of a preferred embodiment of the enclosure 1. As shown in FIG. 6A, the upper portions 10 of the enclosure 1 may be connected together by a foldable material 20. When the enclosure 1 is in its folded, compact configuration the material 20 which connects the upper portions 10 of the enclosure 1 may be folded between the panels 13. FIG. 6B shows how the material 20 may be unfolded before or during inflation of the enclosure 1. The material 20 may further comprise relatively rigid portions 21 which may remain folded between the panels 13 until the enclosure 1 is ready to be used. The rigid portions 21 are preferably provided attached and extending from the lower edges of the material 20 and the lower edges of the upper portions 10 of the enclosure 1. FIG. 6C shows the relatively rigid portions 21 in their unfolded state. The ridged portions 21 may be unfolded before or after inflation of the enclosure 1 and provide means for stabilising the enclosure 1 structure when unfolded and/or may provide legs for the enclosure 1. Preferably, the rigid portions 21 when unfolded are biased outwardly to maintain the enclosure 1 in its unfolded configuration.

FIG. 7 shows a cross-section through an embodiment of the operating enclosure 1 when in place over a patient 2. It can be seen that in this embodiment the lower side regions of the enclosure 1 may over-hang the sides of the patient 2. This portion of the enclosure 1 may also overhang the operating table and may provide a region 22 into which waste material may be stored.

In use various interfaces and/or ports 4 may be connected to the operating enclosure 1, for example, entrance/exit ports 4 for the surgeon or operating team and peripheral enclosures 6 containing gases, fluids or instruments and tools.

FIGS. 8A-8C show an embodiment in which side compartments 14 are provided on the enclosure 1. Referring to FIG. 8A, the side compartments 14 preferably comprise a rupturable, flexible material which may be single or double folded to defined pleats which enable easy attachment of entrance/exit ports 4. FIGS. 8B and 8C show how the one or more folds in the side compartments 14 enable the entrance/exit ports 4 to be attached relatively easily with one hand whilst applying a counter-pressure with the other hand. The interior of the compartment is sterile, and the entrance/exit port 4 is provided with a bactericidal adhesive to secure it to the compartment surface, whereby sterility can be maintained when the membrane defining the compartment which is engaged with the entrance/exit port 4 is severed to provide access to the interior. The membrane wall on the interior of the compartment can then be severed to gain access to the operating enclosure without contaminating it.

In order to perform operations inside the enclosure 1 one or more, and usually at least two, glove carriers 5 are attached to the outside of the enclosure 1. However, it is often difficult for a user to fit such glove carriers over both hands. In particular after the first glove carrier has been fitted on one hand it obstructs that hand fitting a glove carrier over the other hand.

FIG. 9 shows an embodiment of a preferred glove carrier 5 before it has been attached to the enclosure 1. Glove carriers 5 comprise a glove portion 25 and an enclosing membrane 26 which extends from the cuff of the glove portion 25 and around the outside of the glove portion 25 to form the glove carrier 5. Thus, a surgeon is free to put his hand into the glove portion 25 whilst the eventual outer surface of the glove portion 25 remains sealed and sterile. The enclosing membrane 26 of the glove carrier 5 preferably comprises at least one exterior thumb and/or finger compartment 27 protruding from the outside of the glove carrier 5. This exterior compartment 27 assists the user in fitting a pair of glove carriers 5 over both hands.

In use, after one hand has been fitted into the glove portion 25 of a first glove carrier 5, the user may extend their thumb and/or any number of fingers into the one or more exterior compartments 27 of the first glove carrier 5. In this manner the user is able to use their first hand to grip the second glove carrier 5 between the exterior compartments 27 or between exterior compartment 27 and the enclosing membrane 26 of the first glove carrier 5. As such, once one hand of the user has been fitted with a first glove carrier 5 it is relatively easy for the user to use that hand to assist the fitting of a second glove carrier 5 on the other hand. Furthermore, the exterior compartment(s) 27 of the second glove carrier 5 also provide a portion which can be gripped to help pull the second glove carrier 5 over the second hand. The exterior compartments 27 also enable the user to perform other actions whilst wearing the glove carriers 5, for example, picking up objects or sterilising surfaces outside of the enclosure. At this stage the eventual outer surface of the gloves (which will eventually contact the patient) remain sterile.

It is contemplated herein that any number of exterior compartments 27 for the fingers or thumb of the user may be provided on the exterior surface of the glove carrier 5.

The glove carriers can then be attached to the enclosure 1, or to one of the side compartments 14, at a forward attachment region 28 distal from the cuff or proximal to the fingers of the glove portion 25, and the user may then enter the enclosure 1. At this point the fingers and thumbs of the user must be retracted from the exterior compartments 27 such that the hand can pass through the attachment region 28 and hence enter the enclosure 1 with the glove portion 25 acting as a barrier between the hand and the atmosphere of the enclosure 1.

The attachment region 28 of the glove carrier 5 preferably comprises an initially sealed opening surrounded by an adhesive or adhesive tape, impregnated with a bactericidal substance. The opening may be sealed e.g. with a rupturable membrane (not shown) and the adhesive tape may have a removable backing tape (not shown) over it. Once the membrane is ruptured, the hand and glove can be moved into the enclosure with the glove surface remaining sterile. In addition, this end of the glove carrier 5 may be provided with a resealable seal inwardly of the opening such that the opening can be resealed once the membrane has been broken. Hence the glove carrier 5 could be detached from the enclosure if necessary without compromising the sterility of the glove portion 25.

In order to perform the operating procedure it may be necessary to enter various sterile objects into the operating enclosure 1. These objects may be contained in and kept sterile by a peripheral enclosure 6 which may be attached to the operating enclosure 1 at any stage. The surfaces between the operating 1 and peripheral 6 enclosures may then be cut through to gain access to the objects.

FIG. 10 shows an embodiment of a peripheral enclosure 6. The peripheral enclosure 6 comprises a container region 29 which may be a flexible tubular sack and a joining region. The portion of the peripheral enclosure 6 which is circled shows an enlarged view of the joining region. Preferably, the joining region comprises a protective layer 30 and a sterilising layer 31. In order to attach the peripheral enclosure 6 to the operating enclosure 1 the protective layer 30 is removed. The peripheral enclosure 6 may then be joined to the operating enclosure 1 in the region of the sterilising layer 31.

The sterilising layer 31 is intended to maintain the joining region of the peripheral enclosure 6 and any of its contents sterile during its attachment to the operating enclosure 1. In one embodiment, the sterilising layer 31 comprises a sponge layer which is impregnated with a bactericidal agent, for example, iodine. Alternatively, or in addition, the sterilising layer may comprise a film preferably having a bactericidal substance on the surface which in use contacts the enclosure 1. The joining region may further comprise an adhesive or bactericidal adhesive for attaching the peripheral enclosure 6 to the operating enclosure 1. These adhesives may be provided around the periphery of the joining region. In this embodiment the region of contact between the peripheral enclosure 6 and operating enclosure 1 is sterilised during the attachment of the peripheral enclosure 6, i.e. by the bactericidal substance impregnated in the sponge and/or film layer and/or adhesive. Once the peripheral enclosure 6 and operating enclosure 1 are attached the region of join or the region within the joined portions may be cut through in order to gain access to the peripheral enclosure 6. In this manner tools, instruments organs or any other objects may be removed from or introduced into the peripheral enclosure 6.

In one embodiment the peripheral enclosure comprises a flange portion 32 which extends radially outward from the container portion 29 in the joining region of the peripheral enclosure 6. The flange portion 32 may be a relatively rigid material and is preferably provided with the adhesive or bactericidal adhesive. In this embodiment the sponge and/or film having a bactericidal agent may seal an opening in the end of the container region 29 inward of the flange portion 32.

In order to perform an operation, an operating enclosure 1 is preferably attached to the patient 2 via an operation dock/drape 3. In use, the area around the incision that is intended to be made to perform the operation is prepared by treating it with a bactericidal substance. As quickly as possible, an operation dock/drape 3 is then placed over the area such that it covers the intended incision region. The incision area is thus isolated and should remain sterile. The enclosure 1 may then attached to the operation dock/drape 3.

FIGS. 11A and 11B show preferred embodiments of an operation dock/drape 3,3′. Referring to FIG. 11A, the operation dock/drape 3 may be formed from a docking layer which has a bactericidal adhesive on at least a portion of both the lower and upper surfaces which in use contact the patient 2 and operating enclosure 1 respectively. Most preferably, the bactericidal adhesive is provided on the lower side of the operation dock/drape in the area which in use covers the area of the patient 2 where the incision is to be made. The bactericidal adhesive is also preferably provided on the corresponding portion of the upper surface of the operation dock/drape 3 above the intended area of incision. In this manner the operating enclosure 1 may be fixedly attached to the patient 2 via the operation dock/drape 3 whilst simultaneously sterilising the surfaces therebetween. Thus, when it is desired to make the incision in the patient the wall of the operating enclosure 1, the operating dock/drape 3 and the patient 2 may be cut through without rendering the incision area of the patient 2 susceptible to infection.

In one embodiment, the docking layer may be relatively rigid and may have an opening which in use is placed over the intended incision area of the patient 2. In the preferred embodiment the opening preferably comprises a sponge 35 impregnated with a bactericidal substance. The opening in the docking layer may alternatively, or in addition, be covered by one or more film layers for example, a film layer may be provided on the upper and/or lower surface of the docking layer to cover the opening or sponge 35. The film layer may also be provided with a bactericidal substance. In this embodiment, in use, the sponge 35 or film layer(s) may serve to sterilise the region of contact between the operation dock/drape 3 and the patient 2 where the incision is intended to be made and the corresponding region of the operating enclosure 1.

FIG. 11B shows an enlarged portion of the outer region of an embodiment of an operation dock/drape 3 wherein the outer region comprises separate docking 36 and patient 37 layers. In use the docking layer 36 is attached to the operating enclosure 1 and is preferably a relatively rigid material and the patient layer 37 is adhered to the patient 2 and may comprise a flexible drape material. This embodiment enables the operation dock/drape 3 to provide a rigid docking layer 36 for providing stable attachment of an enclosure 1 whilst also providing a lower flexible patient layer 37 which readily conforms to the patient 2. Accordingly, the enclosure 1 is able to move or flex slightly relative to the patient 2 without pulling the patient layer 37 away from the patient 2. As such, the external atmosphere is prevented from contaminating the incision region of the patient 2.

Preferably, the upper and lower surfaces of the operation dock/drape 3 are provided with protective covering layers (now shown) which are removed prior to attachment to the patient 2 and operating enclosure 1.

FIG. 12 shows another embodiment of a preferred operation dock/drape 3′ similar to that described above in relation to FIG. 11A. In this embodiment the operation dock/drape 3′ comprises an upper cover 38, a disinfecting layer 39, a layer having an opening 40 and an lower cover 41. In use the lower protective cover 41 may be removed and the operation dock/drape 3′ placed on the patient 2 with the opening in layer 40 arranged over the intended area of incision. The inner region of the disinfecting layer 39 preferably comprises a sponge 35′ impregnated with a bactericidal substance. In use the sponge 35′ protrudes through the opening in layer 40 and contacts the intended incision region of the patient 2 to sterilise that area. Once attached to the patient 2 the upper cover 38 may be removed. Preferably, an operating enclosure 1 is then attached to the operation dock/drape 3′. Preferably, at least part of the disinfecting layer 39 serves to sterilise the portion of the operating enclosure 1 which is directly above the intended incision region. Once the operation dock/drape 3 is attached to the patient 2, the enclosure 1 and operation dock/drape 3′ may be cut through to gain access to the incision region. In the preferred embodiment, this entails cutting through the sterilising sponge to communicate the operating environment with the patient 2.

It is also contemplated herein that the operation dock/drape 3′ may be attached to the operating enclosure 1 prior to being attached to the patient 2. In this method the lower cover 41 remains attached to the layer having the opening 40 until immediately prior to connecting the operation dock/drape 3′ and enclosure 1 to the patient 2.

Although the present invention has been described with reference to preferred embodiments and arrangements described for illustrative purposes only, it will be understood by those skilled in the art that various changes in form and detail may be made without departing from the scope of the invention as set forth in the accompanying claims.

1. An inflatable sterile enclosure comprising a flexible membrane and at least a pair of rigid or semi-rigid panels which are arranged to pivot outwardly relative to one another on each side of the enclosure as it is inflated, wherein at least one of the panels is transparent and provides a window.

2. An enclosure as claimed in claim 1, wherein the panels in said at least one pair of panels are hinged together.

3. An enclosure as claimed in claim 1, wherein the panels in said at least one pair of panels are spaced apart on a flexible portion of the enclosure.

4. An enclosure as claimed in claim 1, wherein a plurality of panels are provided on each side of the enclosure.

5. An enclosure as claimed in claim 1, wherein said panels are arranged on the enclosure such that they can be folded to a position lying generally one above the other with the flexible membrane located therebetween.

6. An enclosure as claimed in claim 1, wherein the flexible membrane is in the form of a collapsible bubble.

7. An enclosure as claimed in claim 1, wherein the flexible membrane is transparent.

8. An enclosure as claimed in claim 1, further comprising a gas entry port through which gas or air may be passed in use to inflate the enclosure.

9. An enclosure as claimed in claim 8, further comprising a sterilising filter for sterilising the air or gas in use.

10. An enclosure as claimed in claim 1, further comprising a source of pressurised air or gas arranged in communication with the interior of the enclosure for inflating the enclosure in use.

11. An enclosure as claimed in claim 1, further comprising elongate support members mounted and arranged on the flexible membrane so as to provide a supporting frame for the enclosure once it is inflated.

12. An enclosure as claimed in claim 11, wherein the support members are configured to be interconnectable with one another to form a support frame after inflation of the enclosure.

13. A collapsible, sterile enclosure comprising a flexible membrane which can be inflated to define the interior of the enclosure, there being a plurality of elongate support members secured to the membrane, said members being arranged to define a supporting frame for the enclosure when erected.

14. A collapsible, sterile enclosure comprising a flexible membrane which can be inflated to define an interior of the enclosure, the membrane being provided with one or more inflatable support members, said members being arranged to define a supporting frame for the enclosure when erected.

15. A closed, generally tubular sterile enclosure adapted to form a peripheral enclosure to a large sterile enclosure, comprising an end wall member having means for joining it to a wall of a larger enclosure, wherein a surface on or adjacent to the end wall member is provided with a bactericidal substance which sterilises the join formed between the enclosures in use, the peripheral enclosure containing one or more surgical instruments.

16. A tubular sterile enclosure as claimed in claim 15, wherein the bactericidal substance is covered by a removable protective layer.

17. A tubular sterile enclosure as claimed in claim 15, wherein the joining means comprises an adhesive.

18. A tubular sterile enclosure as claimed in claim 17, wherein the adhesive is provided on the end wall member of the peripheral enclosure as an adhesive tape or layer.

19. A tubular sterile enclosure as claimed in claim 17, wherein the adhesive has a removable backing layer.

20. A tubular sterile enclosure as claimed in claim 17, wherein the joining means comprises an adhesive provided on a flange portion which extends radially outward from the main body of the peripheral enclosure.

21. A tubular sterile enclosure as claimed in claim 15, wherein the end wall member of the peripheral enclosure within the joining means comprises a sponge material impregnated with a bactericidal substance.

22. A tubular sterile enclosure as claimed in claim 15, wherein the joining means or the region within the joining means comprises a perforable membrane.

23. A tubular sterile enclosure as claimed in claim 22, wherein the joining means comprises a membrane having a cleft sealed by a weaker membrane.

24. A peripheral enclosure having a sterile interior adapted to be securable to a sterile operating enclosure and to permit communication between the enclosures without contamination, a connecting surface of the peripheral enclosure being provided with a sponge material impregnated with a bactericidal substance and arranged to sterilise interengaged portions of the enclosures in use.

25. An operating enclosure having a flexible membrane wall, there being a closed attachment compartment of flexible material secured to the wall, the interior of the compartment being sterile, the compartment being pleated to enable pressure to be manually applied on a reverse face of the compartment and transmitted to its front face, whereby an item can be engaged under pressure with the front face and adhered thereto, without the need to access the sterile interior to apply such pressure.

26. A surgical dock or drape having an upper surface and a lower surface, at least a portion of both the upper and lower surfaces comprising a bactericidal adhesive or adhesive tape for joining the surgical dock or drape both to a patient and to an overlying operating enclosure in use.

27. A surgical dock or drape provided with means having a bactericidal substance arranged to sterilise in use regions of an underlying patient and of an operating enclosure overlying the dock or drape.

28. A surgical dock or drape as claimed in claim 27, comprising a layer in which the central portion is a sponge impregnated with a bactericidal substance and arranged to engage both the patient and the operating enclosure in use.

29. A surgical dock or drape as claimed in claim 28, wherein an outer portion of the surgical dock or drape surrounding the sponge comprises an adhesive or bactericidal adhesive on either or both of the surfaces which contact the enclosure and patient in use.

30. A surgical dock or drape as claimed in claim 27, wherein an outer portion of the surgical dock or drape comprises separate docking and patient layers for adhering to the operating enclosure and patient respectively in use.

31. A surgical dock or drape as claimed in claim 30, wherein the docking layer is rigid and the patient layer is flexible so that it can conform to the patient in use.

32. A surgical dock or drape as claimed in claim 27, wherein the means having a bactericidal substance is a membrane layer which seals an opening in the surgical dock or drape and wherein the membrane layer comprises a bactericidal adhesive or adhesive tape.

33. A surgical dock or drape as claimed in claim 27, further comprising an inflatable portion which is arranged to surround the area of incision in a patient in use.

34. A surgical dock or drape as claimed in claim 33, wherein the inflatable portion comprises an adhesive or bactericidal adhesive.

35. A glove carrier comprising a sterile glove region enclosed by a flexible membrane, wherein the flexible membrane comprises at least one exterior thumb or finger compartment alignable in use with a thumb or finger portion of the glove enclosed within the membrane.

36. A glove carrier as claimed in claim 35, wherein the exterior of the flexible membrane is provided with joining means proximal to the fingers of the glove portion so as to enable the flexible membrane of the glove carrier to be attached to an operating enclosure in use.
